(19) United States
(12) Patent Application Publication
(10) Pub. No.: US 2023/0405436 A1
(43) Pub. Date: Dec. 21, 2023

(54) BODY SHAPE MANAGEMENT SYSTEM, ITS CONTROL METHOD, AND CONTROL PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Hiroshi SHIMADA, Tajimi-shi (JP); Atsushi HATANO, Setagaya-ku (JP); Yumi KOYAMA, Shibuya-ku (JP)

(73) Assignee: TOYOTA JIDOSHA KABUSHIKI KAISHA, Toyota-shi (JP)

(21) Appl. No.: 18/337,102

(22) Filed: Jun. 19, 2023

(30) Foreign Application Priority Data
Jun. 20, 2022 (JP) 2022-099189

(51) Int. Cl. A63B 71/06; G06T 11/00; G06T 7/50; A63B 24/00; G16H 50/70

(52) U.S. Cl. CPC A63B 71/0622; G06T 11/00; G06T 7/50; A63B 24/0075; G16H 50/70; G06T 2207/30196; G16H 20/30

(57) ABSTRACT

A body shape management system includes: a predicted body shape calculation unit configured to calculate, for each set period, a predicted body shape of a first user according to a first schedule of the first user stored in a database, the predicted body shape being a body shape estimated by predicting a change from a reference body shape of the first user; and a display control unit configured to display an avatar of the first user along with the first schedule on a monitor, the avatar representing the predicted body shape of the first user calculated for each set period by the predicted body shape calculation unit.

### CROSS REFERENCE TO RELATED APPLICATIONS

This application is based upon and claims the benefit of priority from Japanese patent application No. 2022-099189, filed on Jun. 20, 2022, the disclosure of which is incorporated herein in its entirety by reference.

### BACKGROUND

The present disclosure relates to a body shape management system, its control method, and a control program.

Japanese Unexamined Patent Application Publication No. 2007-310632 discloses an avatar health and body shape prediction system capable of displaying an avatar of a user and predicting his/her own body shape and health condition that he/she will have after a lapse of an arbitrary period.

### SUMMARY

It has been desired to appropriately manage a schedule of a user for improving his/her body shape.

The present disclosure has been made in view of the above-described situation, and an object thereof is to provide a body shape management system, its control method, and a control program capable of appropriately managing a schedule of a user for improving his/her body shape by visually expressing a predicted body shape of the user that he/she will have when he/she behaves according to the schedule.

A body shape management system according to the present disclosure includes: a predicted body shape calculation unit configured to calculate, for each set period, a predicted body shape of a first user according to a first schedule of the first user stored in a database, the predicted body shape being a body shape estimated by predicting a change from a reference body shape of the first user; and a display control unit configured to display an avatar of the first user along with the first schedule on a monitor, the avatar representing the predicted body shape of the first user calculated for each set period by the predicted body shape calculation unit. The above-described body shape management system can appropriately manage a schedule of a user for improving his/her body shape by visually expressing a predicted body shape of the user that he/she will have when he/she behaves according to the schedule.

The body shape management system may further include an acquisition unit configured to acquire information about an actual body shape of the first user, in which the predicted body shape calculation unit may be configured to use the actual body shape of the first user acquired by the acquisition unit as the reference body shape.

The predicted body shape calculation unit may calculate, for each set period, the predicted body shape, which is the body shape estimated by predicting a change from the reference body shape of the first user, based on at least one of an item representing a type of exercise (hereinafter also referred to simply as an exercise item) to be performed by the first user and a meal of the first user included in the first schedule.

When the first schedule is updated, the predicted body shape calculation unit may calculate, for each set period, the predicted body shape, which is the body shape estimated by predicting a change from the reference body shape of the first user, according to the updated first schedule.

The predicted body shape calculation unit may be configured so as to be able to update the predicted body shape of the first user according to progress made by the first user on the first schedule.

A second schedule of a second user may be further stored in the database; the predicted body shape calculation unit may be further configured to calculate, for each set period, a predicted body shape of the second user according to the second schedule, the predicted body shape being a body shape estimated by predicting a change from a reference body shape of the second user; and the display control unit may display the avatar of the first user along with the first schedule on the monitor and display an avatar of the second user along with the second schedule on the monitor, the avatar of the first user representing the predicted body shape of the first user calculated for each set period by the predicted body shape calculation unit, and the avatar of the second user representing the predicted body shape of the second user calculated for each set period by the predicted body shape calculation unit.

The body shape management system may further include an acquisition unit configured to acquire information about an actual body shape of the first user and information about an actual body shape of the second user, in which the predicted body shape calculation unit may be configured to use the actual body shape of the first user acquired by the acquisition unit as the reference body shape of the first user, and use the actual body shape of the second user acquired by the acquisition unit as the reference body shape of the second user.

The predicted body shape calculation unit may calculate, for each set period, the predicted body shape, which is the body shape estimated by predicting a change from the reference body shape of the first user, based on at least one of an exercise item to be performed by the first user and a meal of the first user included in the first schedule, and the predicted body shape, which is the body shape estimated by predicting a change from the reference body shape of the second user, based on at least one of an exercise item to be performed by the second user and a meal of the second user included in the second schedule.

When the first schedule is updated, the predicted body shape calculation unit may calculate, for each set period, the predicted body shape, which is the body shape estimated by predicting a change from the reference body shape of the first user, according to the updated first schedule; and when the second schedule is updated, the predicted body shape calculation unit may calculate, for each set period, the predicted body shape, which is the body shape estimated by predicting a change from the reference body shape of the second user, according to the updated second schedule.

The predicted body shape calculation unit may be configured so as to be able to update the predicted body shape of the first user according to progress made by the first user on the first schedule, and update the predicted body shape of the second user according to progress made by the second user on the second schedule.

The set period may be changeable.

A method for controlling a body shape management system according to the present disclosure includes: calculating, for each set period, a predicted body shape of a first user according to a first schedule of the first user stored in a database, the predicted body shape being a body shape estimated by predicting a change from a reference body shape of the first user; and displaying an avatar of the first user along with the first schedule on a monitor, the avatar representing the predicted body shape of the first user calculated for each set period by the predicted body shape calculation unit. By the above-described method for controlling a body shape management system, it is possible to appropriately manage a schedule of a user for improving his/her body shape by visually expressing a predicted body shape of the user that he/she will have when he/she behaves according to the schedule.

A control program according to the present disclosure causes a computer to perform: a process for calculating, for each set period, a predicted body shape of a first user according to a first schedule of the first user stored in a database, the predicted body shape being a body shape estimated by predicting a change from a reference body shape of the first user; and a process for displaying an avatar of the first user along with the first schedule on a monitor, the avatar representing the predicted body shape of the first user calculated for each set period by the predicted body shape calculation unit. By the above-described control program, it is possible to appropriately manage a schedule of a user for improving his/her body shape by visually expressing a predicted body shape of the user that he/she will have when he/she behaves according to the schedule.

According to the present disclosure, it is possible to provide a body shape management system, its control method, and a control program capable of appropriately managing a schedule of a user for improving his/her body shape by visually expressing a predicted body shape of the user that he/she will have when he/she behaves according to the schedule.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing an example of a configuration of a body shape management system according to a first embodiment;

FIG. 2 is a flowchart showing operations performed by the body shape management system according to the first embodiment;

FIG. 3 shows an example of contents displayed on a monitor;

FIG. 4 is a block diagram showing an example of a configuration of a body shape management system according to a second embodiment; and

FIG. 5 shows an example of contents displayed on a monitor.

### DESCRIPTION OF EMBODIMENTS

The present disclosure will be described hereinafter through embodiments of the disclosure, but the invention according to the claims is not limited to the below-shown embodiments. Further, all the components/structures described in the embodiments are not necessarily indispensable as means for solving the problem. For clarifying the explanation, the following description and the drawings are partially omitted and simplified as appropriate. The same reference numerals (or symbols) are assigned to the same elements throughout the drawings and redundant descriptions thereof are omitted as appropriate.

### First Embodiment

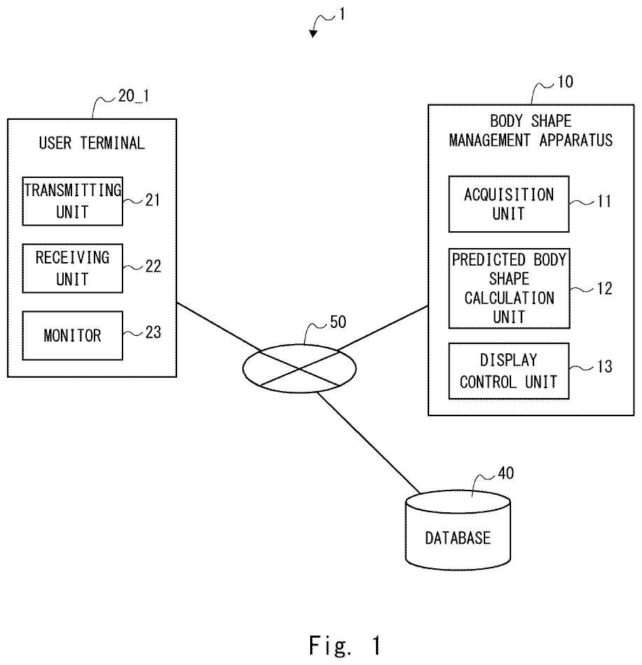

FIG. 1 is a block diagram showing an example of a configuration of a body shape management system 1 according to a first embodiment. The body shape management system 1 displays, for each set period, an avatar (a virtual person) representing a predicted body shape of a user along with a schedule of the user on a monitor. Since the body shape management system 1 can visually express the predicted body shape of the user that he/she will have when he/she behaves according to the schedule, it can appropriately manage the schedule of the user for improving his/her body shape. Details of the body shape management system 1 or the like will be described hereinafter.

As shown in FIG. 1, the body shape management system 1 includes a body shape management apparatus 10, a user terminal 20_1, a database 40, and a network 50. The body shape management apparatus 10 can also be referred to as a body shape management system. The user terminal 20_1, the database 40, and the body shape management apparatus 10 are configured so that they can communicate with each other through the wired or wireless network 50.

The user terminal 20_1 is a terminal owned by or temporarily assigned to a user U1 and capable of performing wireless communication such as a PC (Personal Computer) terminal, a mobile terminal such as a smartphone or a tablet-type terminal, or a dedicated communication terminal provided for the body shape management system. In this embodiment, an example case where the user terminal 20_1 is a mobile terminal such as a smartphone owned by the user U1 will be described.

The user terminal 20_1 includes at least a transmitting unit 21, a receiving unit 22, and a monitor 23. For example, the user U1 enters information about a schedule of the user U1, information about the actual body shape of the user U1, and the like into the user terminal 20_1 by operating an operation screen or the like of the user terminal 20_1. The transmitting unit 21 receives the information about the schedule of the user U1 entered by the operation performed by the user U1, and transmits the received information to the database 40 through the network 50. Further, the transmitting unit 21 receives the information about the actual body shape of the user U1 entered by the operation performed by the user U1, and transmits the received information to the body shape management apparatus 10 through the network 50. The receiving unit 22 receives, through the network 50, information about contents to be displayed, instructed to be displayed on the monitor 23 by the body shape management apparatus 10. The monitor 23 displays the instructed contents to be displayed, received by the receiving unit 22.

Note that the information about the actual body shape of the user U1 means the apparent body shape of the user U1 at the present time (at the time when the information is entered). The information about the actual body shape of the user U1 may be directly entered by the user U1, or may be estimated by the body shape management apparatus 10 or the like based on information about, for example, the age, gender, height, and weight of the user U1 at the present time.

The body shape management apparatus 10 calculates, for each set period, a predicted body shape of the user U1, and displays an avatar representing the calculated predicted body shape of the user U1 along with the schedule of the user U1 on the monitor 23.

Specifically, the body shape management apparatus 10 includes at least an acquisition unit 11, a predicted body shape calculation unit 12, and a display control unit 13.

The acquisition unit 11 acquires information transmitted from the user terminal 20_1. For example, the acquisition unit 11 acquires information about the actual body shape of the user U1. Further, the acquisition unit 11 acquires information stored in the database 40. Note that, for example, information about the schedule of the user U1 is stored in the database 40. Further, information about, for each exercise item, calories that are consumed when the user has performed that exercise item, and information about, for each meal, calories that are taken when the user has eaten that meal are stored in the database 40.

The predicted body shape calculation unit 12 calculates, for each set period, a predicted body shape of the user U1 according to the schedule of the user U1. More specifically, the predicted body shape calculation unit 12 calculates (estimates), for each set period, the total value of consumed calories and that of taken calories from the exercise items and the meals included in the schedule of the user U1, and calculates (estimates), for each set period, a predicted body shape of the user U1 from the calculated total values. Note that the exercise items are not limited to sports such as running, rope jumping, swimming, tennis, soccer, and exercises in a training gym, but also includes, for example, traveling to workplaces and predetermined works.

Note that the predicted body shape of the user U1 is a body shape estimated by predicting changes from the reference body shape of the user U1. Note that the reference body shape of the user U1 is set, for example, by referring to the actual body shape of the user U1. In other words, for example, the actual body shape of the user U1 is used as the reference body shape of the user U1. However, the reference body shape of the user U1 is not limited to those that are set by referring to the actual body shape of the user U1, and an arbitrary body shape may be set as the reference body shape. The unit for the set period can be arbitrarily set, and is, for example, on a weekly basis or on a monthly basis.

Further, when the schedule is updated, the predicted body shape calculation unit 12 recalculates, for each set period, the predicted body shape of the user U1 according to the updated schedule.

Further, the predicted body shape calculation unit 12 may be configured so as to be able to update the predicted body shape of the user U1 according to progress made by the user U1 on the schedule. For example, when the user U1 does not exercise according to the schedule or eats a meal having excessive calories, the predicted body shape calculation unit 12 updates the predicted body shape to a less improved body shape.

The display control unit 13 displays the avatar of the user U1 representing the predicted body shape of the user U1, which has been calculated for each set period by the predicted body shape calculation unit 12, along with the schedule of the user U1 on the monitor 23. Note that the expression that the display control unit 13 displays the avatar and the schedule of the user U1 on the monitor 23 may include processes in which the display control unit 13 outputs the avatar and the schedule of the user U1, and the user terminal 20_1 acquires the avatar and the schedule of the user U1 output from the display control unit 13 and displays the acquired avatar and the schedule on the monitor 23. In this way, the user U1 can visually know his/her predicted body shape that he/she will have when he/she behaves according to the schedule, so that it is possible to encourage the user U1 to improve his/her body shape.

Note that the user U1 can adjust his/her predicted body shape represented by the avatar by adjusting the behaviors (exercise items and meals) included in the schedule. That is, the user U1 can appropriately manage the schedule for improving his/her body shape while checking his/her predicted body shape. Note that an avatar identical to or close to the actual body shape of the user U1 may be used as the avatar of the user U1, or an avatar representing a personified and animated animal or the like may be used as the avatar of the user U1.

As described above, the body shape management system 1 according to this embodiment displays, for each set period, an avatar representing a predicted body shape of the user U1 along with the schedule of the user U1 on the monitor 23. Since the body shape management system 1 can visually express the predicted body shape of the user U1 that he/she will have when he/she behaves according to the schedule, it can appropriately manage the schedule of the user U1 for improving his/her body shape.

### (Operation of Body Shape Management System 1)

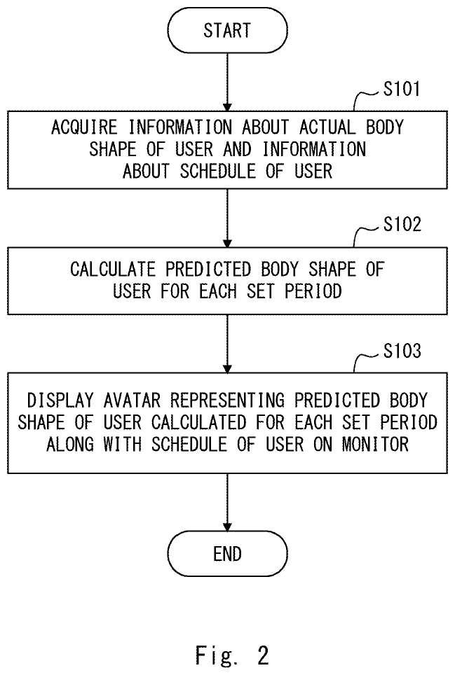

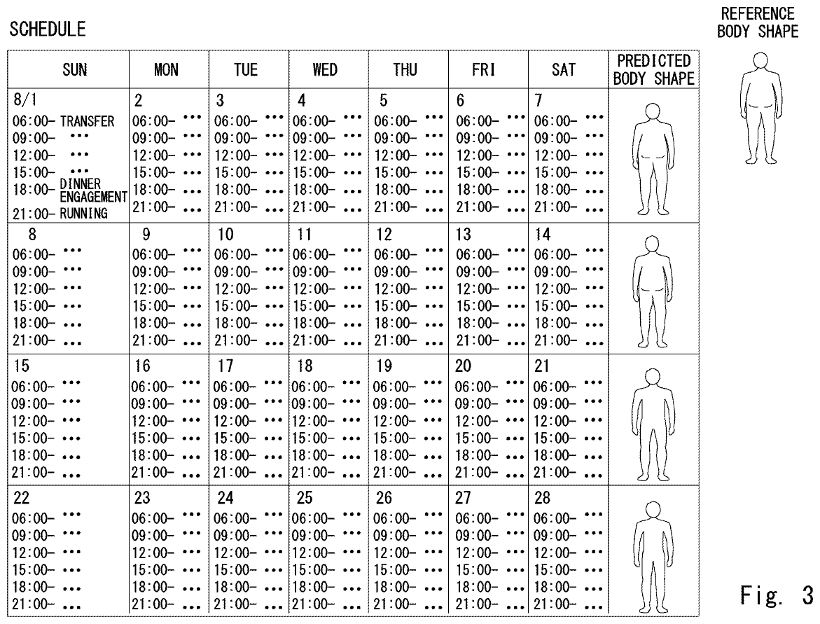

Next, operations performed by the body shape management system 1 will be described with reference to FIGS. 2 and 3. FIG. 2 is a flow chart showing operations performed by the body shape management system 1. FIG. 3 shows an example of contents displayed on the monitor 23 of the user terminal 20_1.

In the body shape management apparatus 10, the acquisition unit 11 acquires, for example, information about the actual body shape of the user U1 transmitted from the user terminal 20_1, and information about the schedule of the user U1 stored in the database 40 (Step S101).

After that, the predicted body shape calculation unit 12 calculates, for each set period, a predicted body shape of the user U1 according to the schedule of the user U1 (Step S102). More specifically, the predicted body shape calculation unit 12 calculates (estimates), for each set period, the total value of consumed calories and that of taken calories from the exercise items and the meals included in the schedule of the user U1, and calculates (estimates), for each set period, a predicted body shape of the user U1 from the calculated total values.

After that, the display control unit 13 displays, for each set period, the avatar of the user U1 representing the predicted body shape of the user U1 calculated by the predicted body shape calculation unit 12 along with the schedule of the user U1 on the monitor 23 (Step S103).

As shown in FIG. 3, a schedule of about one month of the user U1, and four avatars, in total, representing predicted body shapes of the user U1 calculated on a weekly basis by the predicted body shape calculation unit 12 are displayed on the monitor 23 of the user terminal 20_1. Note that an avatar representing the reference body shape of the user U1 is also displayed on the monitor 23 of the user terminal 20_1 for comparison with the avatars representing the respective predicted body shapes of the user U1. As shown in FIG. 3, the predicted body shapes of the user U1 represented by the avatars are gradually improved and become leaner when the user U1 behaves according to the schedule. In this way, the user U1 can visually know his/her predicted body shape that he/she will have when he/she behaves according to the schedule, so that it is possible to encourage the user U1 to improve his/her body shape.

Note that the user U1 can adjust his/her predicted body shape represented by the avatar by changing the behaviors (exercise items and meals) included in the schedule. That is, the user U1 can appropriately manage the schedule for improving his/her body shape while checking his/her predicted body shape.

As described above, the body shape management system 1 according to this embodiment displays, for each set period, an avatar representing a predicted body shape of the user U1 along with the schedule of the user U1 on the monitor 23. Since the body shape management system 1 can visually express the predicted body shape of the user U1 that he/she will have when he/she behaves according to the schedule, it can appropriately manage the schedule of the user U1 for improving his/her body shape.

### Second Embodiment

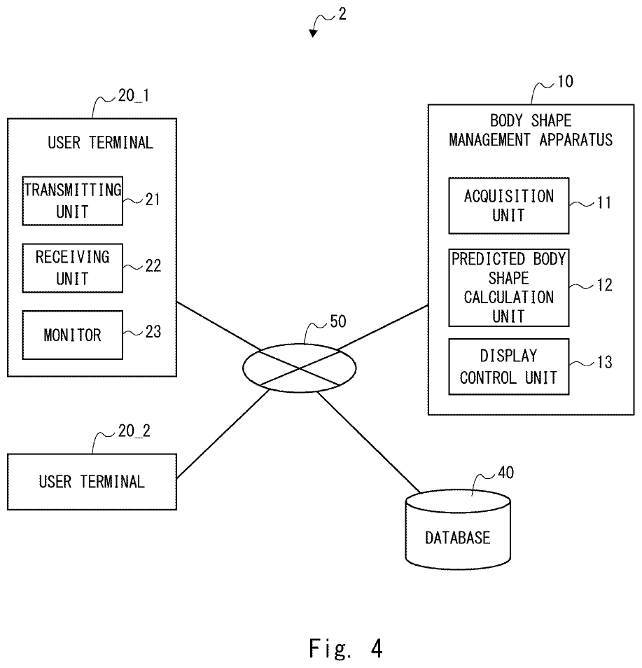

FIG. 4 is a block diagram showing an example of a configuration of a body shape management system 2 according to a second embodiment. While the body shape management system 1 includes only one user terminal 20_1, the body shape management system 2 includes n user terminals 20_1 to 20_n (n is an integer equal to or greater than 2). In this embodiment, an example case where n=2, i.e., where the body shape management system 2 includes two user terminals 20_1 and 20_2 will be described.

As shown in FIG. 4, the body shape management system 2 includes a body shape management apparatus 10, the user terminals 20_1 and 20_2, a database 40, and a network 50. The user terminals 20_1 and 20_2, the database 40, and the body shape management apparatus 10 are configured so that they can communicate with each other through the wired or wireless network 50.

The user terminal 20_2 is a terminal owned by or temporarily assigned to a user U2 and capable of performing wireless communication. In this embodiment, an example case where, similar to the user terminal the user terminal 20_2 is a mobile terminal such as a smartphone owned by user U2 will be described. The specific configuration of the user terminal 20_2 is similar to that of the user terminal 20_1, and therefore the description thereof is omitted.

The body shape management apparatus 10 calculates, for each set period, a predicted body shape of each of users U1 and U2, and displays avatars representing the calculated predicted body shapes of the users U1 and U2, respectively, along with their schedules on the monitors 23 of the user terminals 20_1 and 20_2, respectively.

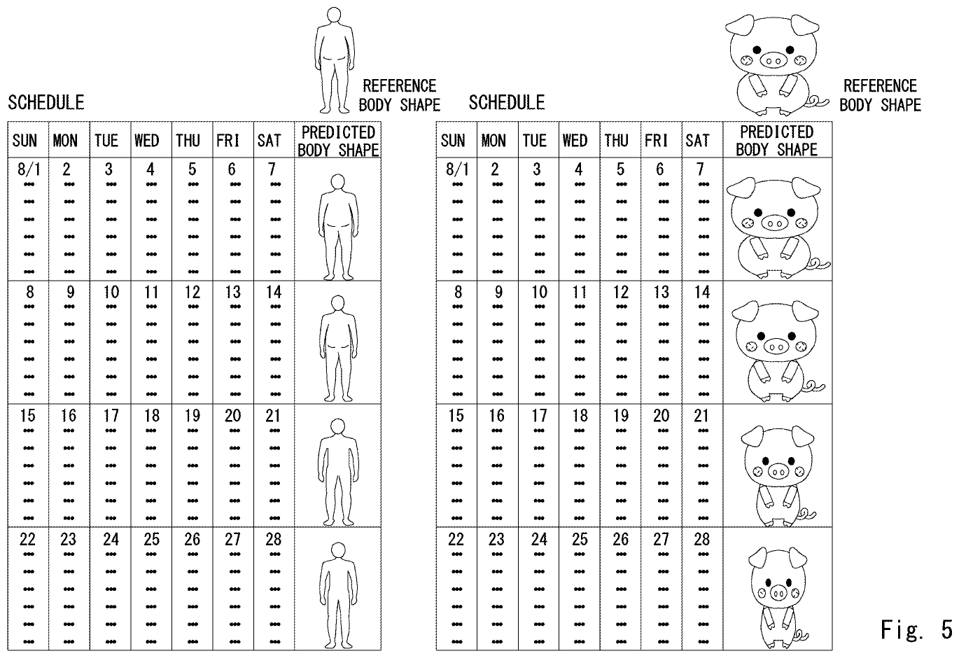

FIG. 5 shows an example of contents displayed on the monitor 23 of each of the user terminals 20_1 and 20_2. As shown in FIG. 5, firstly, a schedule of about one month of the user U1, and four avatars, in total, representing predicted body shapes of the user U1 calculated on a weekly basis by the predicted body shape calculation unit 12 are displayed on (a left part of) the monitor 23 of each of the user terminals. Further, a schedule of about one month of the user U2, and four avatars, in total, representing predicted body shapes of the user U2 calculated on a weekly basis by the predicted body shape calculation unit 12 are displayed on (a right part of) the monitor 23 of each the user terminals. Avatars representing the reference body shapes of the users U1 and U2, respectively, are also displayed on the monitors 23 of each of the user terminals. Further, in the example shown in FIG. 5, an avatar having a body shape close to the actual body shape of the user U1 is used as the avatar of the user U1, and an avatar representing a personified and animated pig is used as the avatar of the user U2. As shown in FIG. 5, the predicted body shapes of the users U1 and U2 represented by the avatars are gradually improved and become leaner when the users U1 and U2 behave according to the schedules. The users U1 and U2 can visually know their predicted body shapes that they will have when they behave according to the schedules, so that it is possible to encourage the users U1 and U2 to improve their body shapes. Further, each of the users U1 and U2 can know an improvement in the body shape of the other user, so that owing to the rivalry between them, it is possible to further encourage them to improve their body shapes.

Note that each of the users U1 and U2 can adjust his/her predicted body shape represented by the avatar by changing the behaviors (exercise items and meals) included in the schedule. That is, each of the users U1 and U2 can appropriately manage the schedule for improving his/her body shape while checking his/her predicted body shape.

As described above, the body shape management system 2 according to this embodiment displays, for each set period, avatars representing predicted body shapes of the users U1 and U2, respectively, along with their schedules on the monitors 23 of the user terminals 20_1 and 20_2, respectively. Since the body shape management system 2 can visually express the predicted body shape of each of the users U1 and U2 that he/she will have when he/she behaves according to the schedule, it can appropriately manage the schedules of the users U1 and U2 for improving their body shapes.

Although an example in which the schedule and the avatar of the user U1 and those of the user U2 are both displayed on the monitor 23 of each of the user terminals 20_1 and 20_2 has been described in this embodiment, the present disclosure is not limited to this example. For example, only the schedule and the avatar of the user U1 may be displayed on the monitor 23 of the user terminal 20_1, and only the schedule and the avatar of the user U2 may be displayed on the monitor 23 of the user terminal 20_2.

Note that the present disclosure is not limited to the above-described embodiments, and they can be modified as appropriate without departing from the scope and spirit of the disclosure. Further, the present disclosure may be implemented by combining the above-described embodiments with each other as appropriate.

Further, in the present disclosure, some or all of the control processes performed in the body shape management system can be implemented by having a CPU (Central Processing Unit) execute a computer program.

In the aforementioned program includes instructions (or software codes) that, when loaded into a computer, cause the computer to perform one or more of the functions described in the embodiments. The program may be stored in a non-transitory computer readable medium or a tangible storage medium. By way of example, and not a limitation, the non-transitory computer readable media or tangible storage media can include a RAM (Random-Access Memory), a ROM (Read-Only Memory), a flash memory, an SSD (Solid-State Drive) or other types of memory technologies, a CD-ROM, a DVD (Digital Versatile Disc), a Blu-ray (Registered Trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, the transitory computer readable media or communication media can include electrical, optical, acoustical, or other forms of propagation signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A body shape management system comprising:
a predicted body shape calculation unit configured to calculate, for each set period, a predicted body shape of a first user according to a first schedule of the first user stored in a database, the predicted body shape being a body shape estimated by predicting a change from a reference body shape of the first user; and
a display control unit configured to display an avatar of the first user along with the first schedule on a monitor, the avatar representing the predicted body shape of the first user calculated for each set period by the predicted body shape calculation unit.

2. The body shape management system according to claim 1, further comprising an acquisition unit configured to acquire information about an actual body shape of the first user, wherein
the predicted body shape calculation unit is configured to use the actual body shape of the first user acquired by the acquisition unit as the reference body shape.

3. The body shape management system according to claim 1, wherein the predicted body shape calculation unit calculates, for each set period, the predicted body shape, which is the body shape estimated by predicting a change from the reference body shape of the first user, based on at least one of an exercise item to be performed by the first user and a meal of the first user included in the first schedule.

4. The body shape management system according to claim 1, wherein when the first schedule is updated, the predicted body shape calculation unit calculates, for each set period, the predicted body shape, which is the body shape estimated by predicting a change from the reference body shape of the first user, according to the updated first schedule.

5. The body shape management system according to claim 1, wherein the predicted body shape calculation unit is configured so as to be able to update the predicted body shape of the first user according to progress made by the first user on the first schedule.

6. The body shape management system according to claim 1, wherein
a second schedule of a second user is further stored in the database,
the predicted body shape calculation unit is further configured to calculate, for each set period, a predicted body shape of the second user according to the second schedule, the predicted body shape being a body shape estimated by predicting a change from a reference body shape of the second user, and
the display control unit displays the avatar of the first user along with the first schedule on the monitor and displays an avatar of the second user along with the second schedule on the monitor, the avatar of the first user representing the predicted body shape of the first user calculated for each set period by the predicted body shape calculation unit, and the avatar of the second user representing the predicted body shape of the second user calculated for each set period by the predicted body shape calculation unit.

7. The body shape management system according to claim 6, further comprising an acquisition unit configured to acquire information about an actual body shape of the first user and information about an actual body shape of the second user, wherein
the predicted body shape calculation unit is configured to use the actual body shape of the first user acquired by the acquisition unit as the reference body shape of the first user, and use the actual body shape of the second user acquired by the acquisition unit as the reference body shape of the second user.

8. The body shape management system according to claim 6, wherein the predicted body shape calculation unit calculates, for each set period, the predicted body shape, which is the body shape estimated by predicting a change from the reference body shape of the first user, based on at least one of an exercise item to be performed by the first user and a meal of the first user included in the first schedule, and the predicted body shape, which is the body shape estimated by predicting a change from the reference body shape of the second user, based on at least one of an exercise item to be performed by the second user and a meal of the second user included in the second schedule.

9. The body shape management system according to claim 6, wherein when the first schedule is updated, the predicted body shape calculation unit calculates, for each set period, the predicted body shape, which is the body shape estimated by predicting a change from the reference body shape of the first user, according to the updated first schedule; and when the second schedule is updated, the predicted body shape calculation unit calculates, for each set period, the predicted body shape, which is the body shape estimated by predicting a change from the reference body shape of the second user, according to the updated second schedule.

10. The body shape management system according to claim 6, wherein the predicted body shape calculation unit is configured so as to be able to update the predicted body shape of the first user according to progress made by the first user on the first schedule, and update the predicted body shape of the second user according to progress made by the second user on the second schedule.

11. The body shape management system according to claim 1, wherein the set period is changeable.

12. A method for controlling a body shape management system, comprising:
calculating, for each set period, a predicted body shape of a first user according to a first schedule of the first user stored in a database, the predicted body shape being a body shape estimated by predicting a change from a reference body shape of the first user; and
displaying an avatar of the first user along with the first schedule on a monitor, the avatar representing the predicted body shape of the first user calculated for each set period by the predicted body shape calculation unit.

13. A non-transitory computer readable storage medium storing a control program for causing a computer to perform:
a process for calculating, for each set period, a predicted body shape of a first user according to a first schedule of the first user stored in a database, the predicted body shape being a body shape estimated by predicting a change from a reference body shape of the first user; and
a process for displaying an avatar of the first user along with the first schedule on a monitor, the avatar representing the predicted body shape of the first user calculated for each set period by the predicted body shape calculation unit.
